(19) United States
(12) Patent Application Publication
(10) Pub. No.: US 2006/0116522 A1
(43) Pub. Date: Jun. 1, 2006

(54) Process for the preparation of 5-substituted-1-(4-fluorophenyl)-1,3-dihydroisobenzofurans

(76) Inventors: Ambati Narahari Babu, Karnataka (IN); Vaddamari Srinivas Goud, Karnataka (IN); Santhosh Laxman Gaonkar, Karnataka, IN (US); Sulur G. Manjunatha, Karnataka (IN); Ashok Krishna Kulkarni, Karnataka (IN)

Correspondence Address: SHERIDAN ROSS PC, 1560 BROADWAY, SUITE 1200, DENVER, CO, 80202, US

(21) Appl. No.: 10/526,302

(22) PCT Filed: Aug. 28, 2003
(86) PCT No.: PCT/IN03/00290

§ 371 Date: Oct. 28, 2005

(30) Foreign Application Priority Data
Aug. 29, 2002 (GB) 0220025

(51) Int. Cl. C07D 307/87

(52) U.S. Cl. 549/467

(57) ABSTRACT

The present invention provides a process for the preparation of a 5-substituted-1-(4-fluorophenyl)-1,3-dihydro-isoben-zofuran of Formula (2), an intermediate for the manufacture of citalopram, which process comprises: (a) carrying out a Grignard reaction on a corresponding 5-substituted phthalide of Formula (3) in a co-solvent system, comprising adding (i) prepared 4-fluorophenyl magnesium halide in an ether solvent to (ii) the 5-substituted phthalide in a suitable organic co-solvent to the ether solvent, to form a corresponding 4-substituted-2-hydroxymethyl-4′-fluorobenzophenone of Formula (4); (b) carrying out a ketone reduction of the 4-substituted-2-hydroxymethyl-4′-fluorobenzophenone of Formula (4) following the Grignard reaction, to form a corresponding 4-substituted-2-hydroxymethylphenyl-1-(4-fluorophenyl) methanol of Formula (5); and (c) carrying out a cyclisation reaction on the 4-substituted-2 hydroxymethylphenyl-1-(4-fluorophenyl) methanol of Formula (5) following the reduction reaction, to form said intermediate of Formula (2); wherein R represents Br or CN.

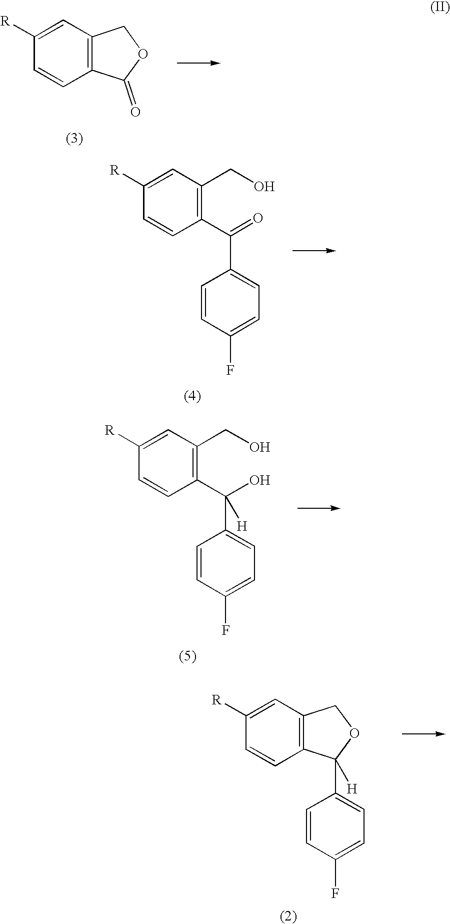

### FIELD OF THE INVENTION

The present invention relates to an improved process for preparation of 5-substituted-1-(4-fluorophenyl)-1,3-dihydro-isobenzofuran (2a, 2b), an important intermediate in the preparation of citalopram, from 5-substituted phthalides.

### BACKGROUND TO THE INVENTION

Citalopram and its pharmaceutically acceptable acid addition salts, such as the hydrogen bromide salt shown in Formula 1 below, described in U.S. Pat. No. 4,650,884, are valuable anti-depressant drugs with few side effects and have been commercially available for a number of years.

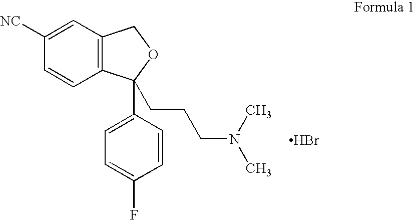

Many processes for the manufacture of citalopram and its acid addition salts are disclosed in the literature, from which it is apparent that 5-substituted phthalanes (5-substituted-1-(4-fluorophenyl)-1,3-dihydroisobenzofurans of Formulae 2a and 2b) are very important key intermediates in the manufacture of citalopram.

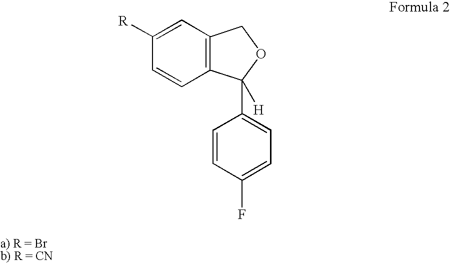

Various processes for the preparation of 5-substituted-1-(4-fluorophenyl)-1,3-dihydroisobenzofurans have been described in the prior art, according to Scheme 1 shown below:

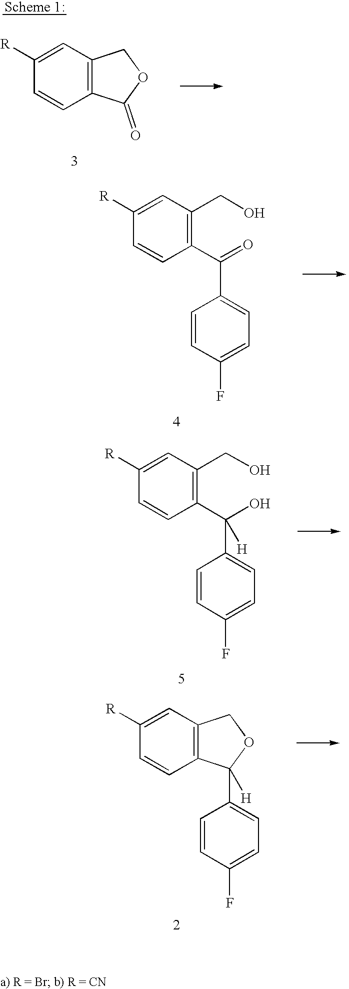

For example, the process described in U.S. Pat. No. 4,136,193 involves the reaction of 4-fluorophenyl magnesium bromide, generated in situ by the reaction of 4-fluorobromobenzene with magnesium in anhydrous diethyl ether solvent medium, with 5-bromophthalide (Formula 3a) in tetrahydrofuran medium. After completion of the reaction, the reaction mass is quenched with aqueous ammonium chloride solution, followed by work-up to provide the intermediate 2-hydroxymethyl-4-bromo-4-fluorobenzophenone (hydroxymethyl-ketone of Formula 4a). The hydroxymethylketone (4a) is then reduced with lithium aluminium hydride in ether medium to provide 4-bromo-2-hydroxymethylphenyl-(4-fluorophenyl)methanol (diol of Formula 5a). The diol (5a) is then cyclised with aqueous phosphoric acid to produce 5-bromophthalane (5-bromo-1-(4-fluorophenyl)-1,3-dihydroisobenzofuran, 2a) which is then converted to 5-cyanophthalane (5-cyano-1-(4-fluorophenyl)-1,3-dihydroisobenzofuran, 2b) by reaction with cuprous cyanide.

The main drawback of this process is in the handling of diethyl ether at plant level. Diethyl ether is a highly volatile, inflammable solvent having a very low flash point. Hence, efficient recovery and recycling of the solvent at the commercial level is not possible. Furthermore, the handling of lithium aluminium hydride, a highly pyrophoric, moisture-sensitive material, is also very difficult at plant level. Therefore, the process is not commercially attractive.

U.S. Pat. No. 6,291,689 discloses a process for preparing 5-cyanophthalane (5-cyano-1-(4-fluorophenyl)-1,3-dihydroisobenzofuran, 2b) in which a solution of 4-fluorophenyl magnesium bromide, prepared from 4-bromofluorobenzene and magnesium turnings in dry tetrahydrofuran, is added drop-wise to a suspension of 5-cyanophthalide (3b) in dry tetrahydrofuran below 5° C. After the addition is completed, ethanol is added to the reaction mixture and a large excess of sodium borohydride (2.0 molar equivalents) is added lot-wise to the reaction mixture. The reaction mixture is stirred overnight at room temperature and then about ⅔ of the solvent is removed under vacuum. Water is added to the reaction mixture and the resulting solution is extracted with ethyl acetate. The ethyl acetate is then distilled off under vacuum to provide the crude diol 4-cyano-2-hydroxymethylphenyl-(4-fluorophenyl)methanol (5b) as an oil. The oil is purified by column chromatography to produce the pure diol (5b) as a solid. However, the oil as such is cyclised in the presence of 60% phosphoric acid solution at 80° C. for 3 hours. The acid solution is then extracted twice with toluene and the organic layer is separated. The combined toluene layer is distilled under vacuum to get the oily residue. The oily residue is then crystallized in ethanol to produce the pure 5-cyanophthalane (2b). The overall yield is 29% from 5-cyanophthalide.

The major drawbacks of this process are that:
i) an expensive solvent, anhydrous tetrahydrofuran, is used which, under the reaction work-up conditions, is difficult to recover and recycle, and thus makes the process uneconomical;
ii) different solvents (e.g. ethyl acetate and toluene) are used at different stages and hence the process becomes commercially unattractive; and
iii) a large excess of sodium borohydride is used during the reduction stage, making the process potentially dangerous.

The present invention seeks to address these problems and provides a very simple method according to Scheme 1 for the preparation of pure 5-substituted phthalanes (2a,b) from 5-substituted phthalides (3a,b), without the isolation of any intermediate and with improved yield and quality of the product.

The present invention also provides a simple procedure for the preparation of the diol (5b) of high purity, for example, greater than 97% purity, which, on further cyclisation with a catalytic amount of p-toluenesulfonic acid in an organic solvent, results in 5-cyanophthalane (2b) of similar high purity.

### SUMMARY OF THE INVENTION

According to a first aspect of the present invention, there is provided a process for the preparation of a 5-substituted-1-(4-fluorophenyl)-1,3-dihydro-isobenzofuran of Formula 2, an intermediate for the manufacture of citalopram, which process comprises:
(a) carrying out a Grignard reaction on a corresponding 5-substituted phthalide of Formula 3 in a co-solvent system, comprising adding (i) prepared 4-fluorophenyl magnesium halide in an ether solvent to (ii) the 5-substituted phthalide in a suitable organic co-solvent to the ether solvent, to form a corresponding 4-substituted-2-hydroxymethyl-4′-fluorobenzophenone of Formula 4,
(b) carrying out a ketone reduction of the 4-substituted-2-hydroxymethyl-4′-fluorobenzophenone of Formula 4 following the Grignard reaction, to form a corresponding 4-substituted-2-hydroxymethylphenyl-1-(4-fluorophenyl) methanol of Formula 5, and
(c) carrying out a cyclisation reaction on the 4-substituted-2-hydroxymethylphenyl-1-(4-fluorophenyl) methanol of Formula 5 following the reduction reaction, to form said intermediate of Formula 2: 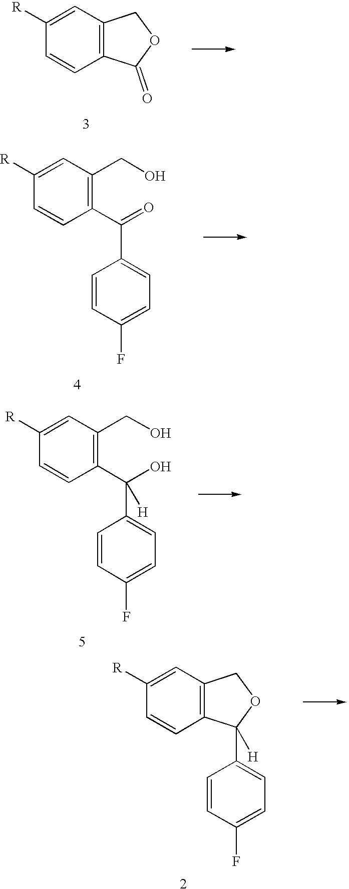
   wherein R represents Br or CN.

Where the 5-substituted-1-(4-fluorophenyl)-1,3-dihydro-isobenzofuran is 5-bromophthalane, the corresponding 5-substituted phthalide is 5-bromophthalide. Where the 5-substituted-1-(4-fluorophenyl)-1,3-dihydro-isobenzofuran is 5-cyanophthalane, the corresponding 5-substituted phthalide is 5-cyanophthalide.

According to a second aspect of the present invention, there is provided a process for preparation of 4-bromo-2-hydroxymethylphenyl-1-(4-fluorophenyl) methanol or 4-cyano-2-hydroxymethylphenyl-1-(4-fluorophenyl) methanol of Formula 5, which process comprises:
(a) carrying out a Grignard reaction on a corresponding 5-substituted phthalide of Formula 3 in a co-solvent system, comprising adding (i) prepared 4-fluorophenyl magnesium halide in an ether solvent to (ii) the 5-substituted phthalide in a suitable organic co-solvent to the ether solvent, to form a corresponding 4-substituted-2-hydroxymethyl-4′-fluorobenzophenone of Formula 4, and
(b) carrying out a ketone reduction of the 4-substituted-2-hydroxymethyl-4′-fluorobenzophenone of Formula 4 with sodium borohydride, to form 4-bromo-2-hydroxymethylphenyl-1-(4-fluorophenyl) methanol or 4-cyano-2-hydroxymethylphenyl-1-(4-fluorophenyl) methanol of Formula 5: 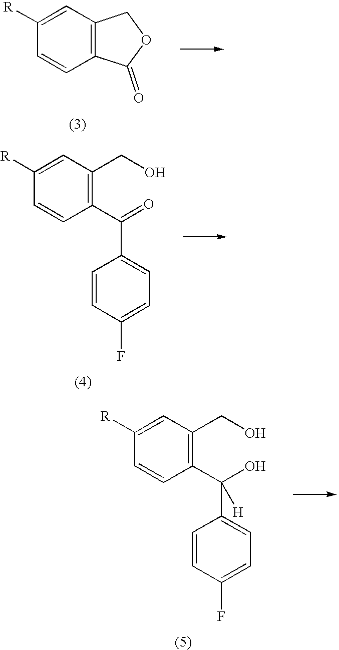
   wherein R represents Br or CN.

If desired, 4-cyano-2-hydroxymethylphenyl-(4-fluorophenyl)methanol may be isolated as a solid directly from the reaction mixture with an HPLC purity of 99%.

### DETAILED DESCRIPTION OF THE INVENTION AND PREFERRED EMBODIMENTS

The process comprises a Grignard reaction, in which the molar ratio of 4-fluorophenyl magnesium halide to the 5-substituted phthalide preferably is 1:1 to 1.4:1.

Although tetrahydrofuran (THF) is the preferred ether solvent, others that may be used include 1,4-dioxane, diethylether or dimethoxyethane.

Preferably, the co-solvent is an aliphatic or aromatic chlorinated solvent or an aromatic hydrocarbon. Where the co-solvent is an aliphatic or aromatic chlorinated solvent, it is suitably selected from methylene dichloride, ethylene dichloride, trichloroethane, carbon tetrachloride, chloroform, chlorobenzene, dichlorobenzene, and mixtures thereof. Methylene dichloride and especially chloroform are particularly preferred. As aromatic hydrocarbon co-solvents, toluene, benzene or xylene, or mixtures thereof, are preferred. Toluene is particularly preferred.

Particularly preferably, the ether solvent and co-solvent are both dry and suitably the volumetric ratio of ether solvent to co-solvent is between 3:10 and 6:7. The lowest proportion of the ether solvent to the co-solvent is restricted by the tendency of the Grignard reagent to precipitate out of solution.

The Grignard reaction is suitably carried out at a temperature of below 10° C., preferably at a temperature from −6° C. to +6° C., and most preferably at a temperature from −6° C. to −2° C.

The process comprises a ketone reduction step following the Grignard reaction. The reducing agent for the reduction step is sodium borohydride. Preferably about 0.25 to about 1.0 molar equivalents of sodium borohydride are used. Particularly preferably only about 0.5 molar equivalents of sodium borohydride are used. This starkly contrasts to the prior art where an excess of sodium borohydride is required.

The process according to the first aspect further comprises carrying out a cyclisation reaction following the reduction reaction. The cyclisation reaction is carried out in presence of an inorganic acid or organic acid. Inorganic acids that may be used include aqueous phosphoric acid and aqueous sulfuric acid, but preferably aqueous hydrochloric acid, more preferably concentrated hydrochloric acid, is used. Organic acids that may be used include methanesulfonic acid, benzenesulfonic acid and para-toluene sulfonic acid (PTSA). A particularly preferred organic acid is PTSA.

The amount of acid used is suitably a limited amount and preferably is a catalytic amount, i.e. not substantially more than the minimum amount required for catalysis of the cyclisation reaction. Where PTSA is used, it is suitably present in a catalytic amount of 5 to 10% w/w with respect to the 5-substituted phthalide.

Advantageously, the entire process according to the first aspect of the present invention, comprising the Grignard reaction, reduction reaction and cyclisation reaction, may be carried out in a reaction vessel, even just one common vessel, without isolation of intermediates from solution.

In a preferred embodiment of the invention, starting from 5-bromophthalide (3a), a solution of 4-fluorophenyl magnesium bromide is prepared from 4-bromofluorobenzene, magnesium turnings and catalytic amount of iodine in dry tetrahydrofuran (THF), and is added drop-wise to a suspension of 5-bromophthalide (3a, 1 molar equivalent) in a dry organic co-solvent under nitrogen atmosphere at a temperature below 10° C., preferably −6° C. to +6° C., and most preferably −6° C. to −2° C., over a period of 4-6 hours.

After the addition is completed, the reaction mixture is quenched with 20% aqueous ammonium chloride solution, and the organic layer is separated and diluted with methanol.

Then, sodium borohydride (0.5-1.0 molar equivalents, preferably 0.5 molar equivalents) is added lot-wise to the reaction mixture at a temperature of below 25° C. and the reaction mixture is further stirred for an additional 2 hours at the same temperature. After the completion of the reaction, water is added and the organic layer is separated. The organic layer is washed with 10% hydrochloric acid solution, water and then concentrated under reduced pressure to obtain an oily residue.

The oily residue is then subjected to a cyclisation reaction in presence of an inorganic acid or organic acid. A particularly preferred organic acid is para-toluene sulfonic acid (PTSA), and this is suitably used in catalytic amounts.

For example, to the oily residue, aqueous hydrochloric acid is added and the mixture is heated to 60-70° C. for 2-3 hours. After the completion of the reaction, the reaction mixture is cooled to room temperature and extracted with an aliphatic or aromatic hydrocarbon, such as n-hexane, cyclohexane, benzene and toluene. The organic layer is washed with dilute sodium hydroxide solution and water. The organic layer is treated with activated charcoal, and concentrated under reduced pressure to provide 5-bromophthalane (2a) having a purity of greater than 85%.

Alternatively and preferably, the oily residue is dissolved in an organic solvent, for example in toluene, and a catalytic amount of p-toluene sulfonic acid (5-10% w/w) is added. The resulting mixture is heated to 85-90° C. and water formed during the reaction is removed continuously by azeotropic distillation. After the completion of the reaction, the reaction mixture is washed with dilute sodium hydroxide solution, water and finally the solvent is removed under reduced pressure to produce 5-bromophthalane (2a).

5-Bromophthalane (2a) can then be converted to 5-cyanophthalane (2b) using known procedures, without any further purification.

In a second embodiment, starting from 5-cyanophthalide (3b), a solution of 4-fluorophenyl magnesium bromide in tetrahydrofuran is added drop-wise over a period of 4-6 hours to a suspension of 5-cyanophthalide (3b, 1 molar equivalent) in a dry organic solvent under nitrogen atmosphere below 10° C. (preferably −6° C. to +6° C., and most preferably −6° C. to −2° C.).

As in the first embodiment above, the dry organic co-solvent may suitably be an aliphatic or aromatic chlorinated solvent such as methylene dichloride, ethylene dichloride, chloroform or chlorobenzene, or may be an aromatic hydrocarbon such as benzene, toluene or xylene.

After the addition is completed, the reaction mixture is quenched with 20% aqueous ammonium chloride solution the organic layer is separated and diluted with methanol. Then sodium borohydride (0.5 molar equivalents) is added lot-wise to the reaction mixture below 25° C. (suitably 15° C. to 20° C.) and the reaction mixture is stirred for additional 4-6 hours. Then the reaction mixture is quenched over water and the organic layer is separated out. The organic layer is then concentrated completely under vacuum to provide a residue, which is used without any further work up for the next stage. Alternatively, the reaction mixture is cooled to below 10° C. and the precipitated solid is filtered to produce pure crystalline 4-cyano-2-hydroxymethylphenyl-(4-fluorophenyl)methanol (5b) with more than 98% purity by HPLC.

The residue/crystalline solid (5b) is taken in an organic solvent such as toluene or methanol, preferably toluene, followed by cyclisation in 30% aqueous hydrochloric acid. After the completion of the reaction, the reaction mass is cooled to 25-30° C. and extracted with toluene. The organic layer is treated with activated carbon and concentrated under reduced pressure. Isopropanol is added to the residue to provide white crystalline 5-cyanophthalane (2b) having a purity of more than 99% by HPLC. The cyclisation may also be carried out in toluene using a catalytic amount of p-toluenesulfonic acid (5-10% w/w with respect to 5-cyanophthalide) to produce 5-cyanophthalane (2b). The overall yield from 5-cyanophthalide to 5-cyanophthalane is 80%.

As indicated in Table 1 and Table 2 below, the present invention establishes that the presence of a co-solvent such as toluene or ethylene dichloride (and also other co-solvents) with the main ether solvent such as tetrahydrofuran yields a better quality of the 5-substituted phthalanes (2a,b).

By the present invention, a single pot procedure has been developed for preparing 5-substituted phthalanes (2a,b) from 5-substituted phthalides (3a,b) without the isolation of any intermediates, suitably using p-toluenesulfonic acid as a catalyst for the cyclisation of the diol (5a,b).

In summary, there are several major advantages of the present invention over the prior art procedures. First, dry tetrahydrofuran is an expensive solvent and is used in large excess in the Grignard reaction in the prior art process. Under the reaction work-up conditions, the recovery and re-use of dry tetrahydrofuran is difficult. In the present invention, the use of tetrahydrofuran can be minimised by employing one or more co-solvents, which are cheap and readily recoverable. Hence the process is made far more commercially attractive. Secondly, with the use of a co-solvent, the intermediates at each stage are easily taken further by simple work-up procedures without the need for isolation or purification of any intermediates.

Furthermore, using the method of the present invention, 0.50 molar equivalents of sodium borohydride is sufficient to reduce the hydroxyketone (4a,b), as opposed to the excess (2.0 molar equivalents) of sodium borohydride used in the prior art processes.

In the final stage of the process, cyclisation with a catalytic amount of acid avoids any large excess of aqueous acidic effluent which is generated by the use of excess acid as described in the prior art.

The following examples serve to further illustrate the present invention:

### EXAMPLE 1

### Preparation of pure 5-Bromo-1-(4-fluorophenyl)-1,3-dihydroisobenzofuran (2a) Using Halogenated Solvents

A solution of 4-fluorophenyl magnesium bromide prepared from 116 g 4-fluoro bromobenzene (0.662 moles), 18.81 g, magnesium turnings (0.78 moles) and 0.05 g Iodine in dry 300 ml tetrahydrofuran, is added to a suspension of 10 g 5-bromophthalide (0.469 moles) in 1000 ml methylene dichloride at −6 to −2° C. After the reaction is completed, the reaction mass is quenched with 100 ml 20% aqueous ammonium chloride solution. The organic layer is separated and diluted with 100 ml of methanol. Slowly, 12 g of sodium borohydride (0.324 moles) is added in lots over a period of one hour at below 25° C., and the temperature is maintained for an additional hour.

The reaction mass is quenched with 200 ml ice water. The organic layer is separated washed with dilute hydrochloric acid (10%, 100 ml) and then with 100 ml water. The organic layer is dried over anhydrous sodium sulfate and concentrated under reduced pressure to produce 4-bromo-2-hydroxymethylphenyl-(4-fluorophenyl)methanol (5a) as an oil. The resulting oil is dissolved in 600 ml of toluene and p-toluenesulfonic acid (10 g) is added. The reaction mixture is heated to reflux and water is removed by azeotropic distillation. After the completion of the reaction the reaction mass is washed with 100 ml of 10% aqueous sodium hydroxide solution, water (100 ml) and dried over anhydrous sodium sulfate. Solvent is removed completely under reduced pressure to get 5-bromo-1-(4-fluorophenyl)-1,3-dihydroisobenzofuran (2a) as a pale yellow oil.

Yield: 95-100 g

HPLC purity: 90-92%

In the same way, other halogenated solvents like chloroform, ethylene dichloride chlorobenzene were used as a co-solvent in place of methylene dichloride to produce 5-bromophthalane. The yield and purity of 5-bromophthalane (2a) made by using these solvents is given in Table 1:

TABLE 1

| | | | 5-Bromo- | |
|---|---|---|---|---|
| | | | phthalane | |
| | Sub- | | purity | |
| Sl. No | phthalide | Solvent mixture | by HPLC | Yield |
| 1 | (5-bromo- | *Tetrahydrofuran (THF) | 80.5% | 56% |
| 2 | phthalide) | THF: Methylene dichloride | 92.5% | 69.3% |
| 3 | | THF: Ethylene dichloride | 86.5% | 65% |
| 4 | | THF: Chloroform | 92.2% | 72.9% |
| 5 | | THF: Toluene | 82.5% | 58.3% |
| 6 | | THF: Chlorobenzene | 78.5% | 58.3% |
| 7 | | THF: Benzene | 82.5% | 58.3% |

*Prior art process

The isolated 5-bromo-1-(4-fluorophenyl)-1,3-dihydroisobenzofuran can be converted to 5-cyanophthalane as per known processes, e.g. that described in U.S. Pat. No. 4,136,193, to provide pure 5-cyanophthalane.

### EXAMPLE 2

### Preparation of pure 5-Cyano-1-(4-fluorophenyl)-1,3-dihydroisobenzofuran (2b) Using Halogenated Solvents

A solution of 4-fluorophenyl magnesium bromide prepared from 153.33 g 4-fluoro bromobenzene (0.876 moles), 25.33 g magnesium turnings (1.055 moles) and 0.05 g iodine in dry 300 ml tetrahydrofuran, is added to a suspension of 100 g 5-cyanophthalide (0.628 moles) in 1000 ml methylene dichloride at −6 to −2° C. and worked up according to the method of Example 1, resulting in a thick semi-solid. This is triturated with 500 ml of isopropyl alcohol (IPA) and cooled to 0-5° C. to provide 5-cyano-1-(4-fluorophenyl)-1,3-dihydroisobenzofuran (2b) as a solid. This solid is filtered and washed with cold 50 ml of IPA.

Yield: 130-140 g

HPLC purity: 99.32%

In the same way other halogenated solvents like chloroform, ethylene dichloride chlorobenzene were used as a co-solvent in place of methylene dichloride to produce 5-cyanophthalane. The yield and purity of 5-cyanophthalane made by using these solvents is given in Table 2:

TABLE 2

| | | | 5-Cyano- | |
|---|---|---|---|---|
| | | | phthalane | |
| | Sub- | | purity | |
| Sl. No | phthalide | Solvent mixture | by HPLC | Yield |
| 1 | (5-cyano- | *Tetrahydrofuran (THF) | 95.6% | 29% |
| 2 | phthalide) | THF: Methylene dichloride | 99.32% | 86% |
| 3 | | THF: Ethylene dichloride | 99.12% | 85.0% |
| 4 | | THF: Chloroform | 99.35% | 86.5% |
| 5 | | THF: Toluene | 97.5% | 70% |
| 6 | | THF: Chlorobenzene | 94.2% | 78% |
| 7 | | THF: Benzene | 93.5% | 78% |

*Prior art process

### EXAMPLE 3

### Isolation of 4-cyano-2-hydroxymethylphenyl-(4-fluorophenyl) methanol (dihydroxy compound 5b)

A solution of 4-fluorophenyl magnesium bromide prepared from 153.33 g 4-fluoro bromobenzene (0.876 moles), 25.33 g magnesium turnings (1.055 moles) and 0.05 g iodine in dry 300 ml tetrahydrofuran, is added to a suspension of 100 g 5-cyanophthalide (0.628 moles) in 1000 ml methylene dichloride at −6 to −2° C. After the reaction is completed, the reaction mass is quenched with 100 ml 20% aqueous ammonium chloride solution. The organic layer is separated and diluted with 100 ml of methanol. Slowly, 12 g of sodium borohydride (0.324 moles) added over a period of one hour at below 25° C., and the same temperature is maintained for 4-6 hours. The mixture is then cooled to 5-10° C., maintained for 2 hours and then the precipitated solid is filtered. The solid is washed with cold water and dried under vacuum below 40° C. to provide pure 4-cyano-2-hydroxymethylphenyl-(4-fluorophenyl)methanol (5b).

Yield: 115-120 g

HPLC purity: 99.2%

### EXAMPLE 4

### Synthesis of 5-Bromo-1-(4-fluorophenyl)-1,3-dihydroisobenzofuran (2a) Using Aromatic Hydrocarbons as Co-Solvent

A solution of 4-fluorophenyl magnesium bromides prepared from 116 g 4-fluoro bromobenzene (0.662 moles), 18.81 g magnesium turnings (0.78 moles) and 0.05 g iodine in dry 300 ml tetrahydrofuran, is added to a suspension of 10 g 5-bromophthalide (0.469 moles) in 1000 ml of toluene at −6 to −2° C. After the reaction is completed, the reaction mass is quenched with 100 ml 20% aqueous ammonium chloride solution. The organic layer is separated and diluted with 100 ml of methanol. Slowly, 12 g of sodium borohydride (0.324 moles) is added in lots over a period of one hour at below 25° C. and the temperature is maintained for additional one hour. The reaction mass is quenched with 200 ml ice water. The organic layer is separated washed with dilute hydrochloric acid (10%, 100 ml) and then with 100 ml water. To the resulting toluene layer, p-toluenesulfonic acid (10 g) is added. The reaction mixture is heated to reflux and water is removed by azeotropic distillation. After the completion of the reaction, the mass is washed with 100 ml of 10% aqueous sodium hydroxide solution, water (100 ml) and dried over anhydrous sodium sulfate. Solvent is removed completely under reduced pressure to provide 5-bromo-1-(4-fluorophenyl)-1,3-dihydroisobenzofuran (2a) as a pale yellow oil.

Weight: 80-85 g

Purity by HPLC: 82.5%

The above-obtained oil is dissolved in 200 ml hexane at 45-50° C. and cooled to 0-5° C., which is maintained for 3-4 hours. The slurry is filtered and washed with 50 ml chilled hexane and dry under reduced pressure.

Weight: 65-70 g

Purity by HPLC: 97.5%

Melting point: 38-40° C.

### EXAMPLE 5

### Synthesis of 5-Cyano-1-(4-fluorophenyl)-1,3-dihydroisobenzofuran (2b) Using Aromatic Hydrocarbons as Co-Solvent

A solution of 4-fluorophenyl magnesium bromide prepared from 153.33 g 4-fluoro bromobenzene (0.876 moles), 25.33 g magnesium turnings (1.055 moles) and 0.05 g iodine in dry 300 ml tetrahydrofuran, is added to a suspension of 10 g 5-cyanophthalide (0.628 moles) in 1000 ml toluene at −6 to −2° C. and worked-up as explained in Example 4 to provide a thick semi-solid. This is triturated with 500 ml of isopropyl alcohol (IPA) and cooled to 0-5° C. to provide 2b as a solid. The solid is filtered and washed with 50 ml of cold IPA.

Dry weight: 105-110 g

Purity by HPLC: 97.5%

1. A process for the preparation of a 5-substituted-1-(4-fluorophenyl)-1,3-dihydroisobenzofuran comprising:
(a) carrying out a Grignard reaction on a 5-substituted phthalide in a co-solvent system, comprising adding 4-fluorophenyl magnesium halide in an ether solvent to a 5-substituted phthalide in an organic co-solvent to the ether solvent, to form a 4-substituted-2-hydroxymethyl-4′-fluorobenzophenone,
(b) carrying out a ketone reduction of the 4-substituted-2-hydroxymethyl-4′-fluorobenzophenone following the Grignard reaction, to form a 4-substituted-2-hydroxymethylphenyl-1-(4-fluorophenyl) methanol, and
(c) carrying out a cyclisation reaction on the 4-substituted-2-hydroxymethylphenyl-1-(4-fluorophenyl) methanol following the reduction reaction, to form a compound having the structure: 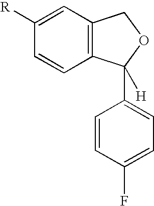
wherein R represents Br or CN.

2. A process according to claim 1, wherein the co-solvent is selected from the group consisting of an aliphatic chlorinated solvent, an aromatic chlorinated solvent and an aromatic hydrocarbon.

3. A process according to claim 2, wherein the co-solvent is an aliphatic or aromatic chlorinated solvent selected from the group consisting of methylene dichloride, ethylene dichloride, trichloroethane, carbon tetrachloride, chloroform, chlorobenzene, dichlorobenzene, and mixtures thereof.

4. A process according to claim 3, wherein the co-solvent is at least one of methylene dichloride and chloroform.

5. A process according to claim 2, wherein the co-solvent is an aromatic hydrocarbon selected from the group consisting of toluene, benzene, xylene, and mixtures thereof.

6. A process according to claim 1, wherein the ether solvent and co-solvent are both dry.

7. A process according to claim 1, wherein the volumetric ratio of ether solvent to co-solvent is between 3:10 and 6:7.

8. A process according to claim 1, wherein the ether solvent is selected from the group consisting of 1,4-dioxane, diethylether, dimethoxyethane and tetrahydrofuran (THF).

9. A process according to claim 1, wherein in the ketone reduction step (b), between 0.25 and 1.0 molar equivalents of sodium borohydride are used as reducing agent.

10. A process according to claim 9, wherein in the ketone reduction step (b), 0.5 molar equivalents of sodium borohydride are used as reducing agent.

11. A process according to claim 1, wherein the cyclisation reaction (c) comprises the use of concentrated hydrochloric acid or an organic acid selected from the group consisting of methanesulfonic acid, benzenesulfonic acid and para-toluene sulfonic acid (PTSA).

12. A process according to claim 11, wherein the acid is used in a catalytic amount.

13. A process according to claim 12, wherein the acid is PTSA in a catalytic amount of 5 to 10% w/w with respect to the 5-substituted phthalide.

14. A process according to claim 1, wherein the Grignard reaction (a) is carried out at a temperature of from −6° C. to −2° C.

15. A process according to claim 1, wherein in the Grignard reaction (a), the molar ratio of 4-fluorophenyl magnesium halide to 5-substituted phthalide is 1:1 to 1.4:1.

16. A process according to claim 1, wherein the entire process, comprising Grignard reaction (a), reduction reaction (b) and cyclisation reaction (c), is carried out in a reaction vessel without isolation of intermediates from solution.

17. A process for preparation of 4-bromo-2-hydroxymethylphenyl-1-(4-fluorophenyl) methanol or 4-cyano-2-hydroxymethylpheny-1-(4-fluorophenyl) methanol comprising:
(a) carrying out a Grignard reaction on a 5-substituted phthalide in a co-solvent system, comprising adding 4-fluorophenyl magnesium halide in an ether solvent to a 5-substituted phthalide in a suitable organic co-solvent to the ether solvent, to form a 4-substituted-2-hydroxymethyl-4′-fluorobenzophenone, and
(b) carrying out a ketone reduction of the 4-substituted-2-hydroxymethyl-4′-fluorobenzophenone with sodium borohydride, to form a compound having the structure: 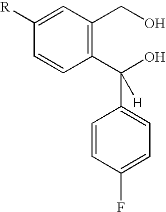
wherein R represents Br or CN.
